(19) United States
(12) Patent Application Publication
(10) Pub. No.: US 2026/0079051 A1
(43) Pub. Date: Mar. 19, 2026

(54) LIGHT MAPPING

(71) Applicant: KORRUS, INC., Los Angeles, CA (US)

(72) Inventor: Eshwar Chandra Bharadwaj SONTI, Wellesley Hills, MA (US)

(21) Appl. No.: 19/402,602

(22) Filed: Nov. 26, 2025

Related U.S. Application Data
(63) Continuation of application No. PCT/US24/31305, filed on May 28, 2024.
(60) Provisional application No. 63/469,104, filed on May 26, 2023.

(51) Int. Cl. G01J 3/28; A61M 21/00; A61M 21/02; A63F 13/65; G01J 1/42

(52) U.S. Cl. CPC G01J 3/2889; A61M 21/02; G01J 1/42; A61M 2021/0044; A63F 13/65; G01J 2001/4266

(57) ABSTRACT

A method of plotting a light map comprising: gathering interior light data for specific locations inside of buildings in a certain region as a function of time; estimating exterior light data for specific locations outside of building is said certain region as a function of time;

wherein said interior and exterior light data includes spectrum and intensity as a function of at least time, and wherein time comprises date and time of day, and wherein accumulated light data comprises said interior and exterior light data; plotting said accumulated light data for said specific locations in said certain region as a function of at least time to create a light map; and wherein said light map provides spectral and intensity data of light as a function of at least time for said specific locations within said certain region.

### CROSS-REFERENCE TO RELATED APPLICATIONS

This application continuation of International Application No. PCT/US24/31305, filed May 28, 2024, which claims the benefit of U.S. Provisional Application No. 63/469,104 filed May 26, 2023, the contents of each of which are incorporated by reference herein in their entirety as if fully set forth herein.

### FIELD OF INVENTION

This disclosure relates to the field of illumination products, and, more particularly, to apparatus and methods for providing people with a platform to measure their light exposure and determine the quantity and quality of additional light needed to maintain healthy circadian rhythms.

### BACKGROUND

Identification of non-visual photoreceptors in the human eye (so-called intrinsically photosensitive retinal ganglion cells, or “ipRGCs”) linked to the circadian system has sparked considerable interest in the effects of various light spectra on health and amenity for human beings. High circadian stimulation may lead to positive effects such as resetting sleep patterns, boosting mood, increasing alertness and cognitive performance, and alleviating seasonal affective depression. However, mis-timed circadian stimulation can also be associated with disruption of the internal biological clock and melatonin suppression, and may be linked to illnesses such as cancer, heart disease, obesity, and diabetes.

Therefore, light plays an important role in peoples' health and wellbeing—on par with the air people breathe and water people drink. In this age of high productivity and urbanization, people need a way to measure their light exposure to determine the light needs for a well-balanced circadian rhythm. Applicant recognizes that there is no platform today that helps people measure the quantity and quality of light exposure they are subjected to from natural and artificial light and the quantity and quality of light of additional light they need. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The present invention provides a platform that helps people measure the quantity and quality of light exposure they are subjected to from natural and artificial light and the quantity and quality of light of additional light they need. Applicant has developed a system and method for mapping light in many different locations, which is referred to herein as the World Light Map (WLM). The WLM is a platform to map the natural and artificial light levels in certain locations all around the world to enable users to get light exposure with circadian recommendations during their daily routine anywhere in the world. In other words, the WLM helps users plan their light exposure around their schedule to retain healthy circadian rhythms.

In one embodiment, the WLM provides a real-time update on the light exposure for an individual at a given time of day and at a given geolocation. Because light is the primary zietberger (external cue) for circadian rhythms, calculating the needed light dose (along with the time and duration of exposure) to deduce the alignment and/or disruption of the user's circadian rhythm is a time series of information. As the modern world demands a user's schedule to be highly productive most often at the expense of user's health and wellbeing over a long period of time, the WLM records, measures and predicts the light needs of users based on their schedule to create awareness and subsequently determine the recommended interventions to maintain regularity in their circadian rhythms.

Accordingly, in one embodiment, the present invention relates to a method of plotting a light map, comprising: (a) gathering interior light data for specific locations inside of buildings in a certain region as a function of time; (b) estimating exterior light data for specific locations outside of building is the certain region as a function of time; (c) wherein the interior and exterior light data includes spectrum and intensity as a function of at least time, and wherein time comprises date and time of day, and wherein accumulated light data comprises the interior and exterior light data; (d) plotting the accumulated light data for the specific locations in the certain region as a function of at least time to create a light map; and (e) wherein the light map provides spectral and intensity data of light as a function of at least time for the specific locations within the certain region.

In one embodiment, the present invention relates to a method of prompting a user to obtain a light requirement, comprising: (a) based on a user's presence in one or more specific locations within a certain region, determining a user's light exposure to light based a light map which provides spectral and intensity data of light as a function of at least time for the specific locations within the certain region; (b) determining a light prescription for the user based at least in part on the user's circadian rhythm; (c) determining the user's light requirement based on the light prescription and the light exposure; and (d) prompting the user to be exposed to the light requirement to satisfy the light prescription.

In one embodiment, the present invention relates to a method of enhancing light in a specific location, comprising: (a) gathering light data for specific locations in a certain region as a function of time; (b) wherein the light data includes spectrum and intensity as a function of at least time, and wherein time comprises date and time of day; (c) plotting the light data for the specific locations in the certain region as a function of at least time to create a light map; (d) wherein the light map provides spectral and intensity data of light as a function of at least time for the specific locations within the certain region; (e) determining a lighting requirement of at least one specific location based on at least a function of the at least one specific location; and (f) recommending a modification in light at the at least one specific location based on the light map and the lighting requirement.

### BRIEF DESCRIPTION OF FIGURES

FIG. 1 shows a schematic of one embodiment of the WLM platform.

FIG. 2 shows a flowchart of one embodiment of the method of the WLM platform.

FIG. 3 shows a flowchart for one embodiment of the Light Crawlers subsystem.

FIG. 4 shows a flowchart for one embodiment of the light engine subsystem.

FIG. 5 shows a flowchart for one embodiment of the light informer subsystem.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

The World Light Map (WLM) helps users plan their light exposure or “light diet” or light requirement around their schedule at the same time retain healthy circadian rhythms. A metric for light diet is important for the usability of the WLM. This metric, in one embodiment, should be intuitive to the user and should incorporate a wide variation of the measurement of light levels. In one embodiment, this metric is reactive to the dynamic evolving environment of light exposure. Applicant proposes herein “Day Light Hours” as this metric which meets all the three parameters. Users worldwide—tropical, subtropics, temperate, frigid zones, technical vs non-technical, developed vs developing nations, multiple languages can relate to “Day Light Hours” and understand it without having it explained. Be it natural light across different weather conditions or artificial light, the light in a wide range of light levels—e.g., traditionally measured in Lumens and Lux—can be mapped on to “Day Light Hours.” Just like how traffic patterns are mapped with a scale of green, orange, red and brick red, “Day Light Hours,” in one embodiment, can be used to derive a scale of the light diet per hour.

Light Diet, like nutrition diet, is both a quantitative and qualitative measure that has prominent consequences on the health and wellbeing. The quantitative aspect of the light diet, in one embodiment, can be addressed with the “Day Light Hours,” analogous to how the quantitative aspect of nutrition diet is articulated using “Calories.” The qualitative aspect of the light diet uses the spectral content of light (e.g., spectral power distribution) analogous to the Nutrition Chart on food items indicating the share of Protein, Fat, Carbohydrates, Fiber content contributing to the calorific value.

In one embodiment, the WLM provides a real-time update on the light exposure for an individual at a given time of day and at a given geolocation. The WLM records, measures and predicts the light diet of users based on their schedule to create awareness and subsequently determine the recommended interventions to maintain regularity in their circadian rhythms.

The WLM is also a platform to capture the light diet for a given space and for its purpose. Light Diet for a space, like the light diet for humans, is a quantitative and qualitative measure. Quantitative measure is often captured in building regulations in terms of candles, lux and/or watts per sf. Qualitative measure is captured using spectral content which is important to ensure the viewing experience of the space.

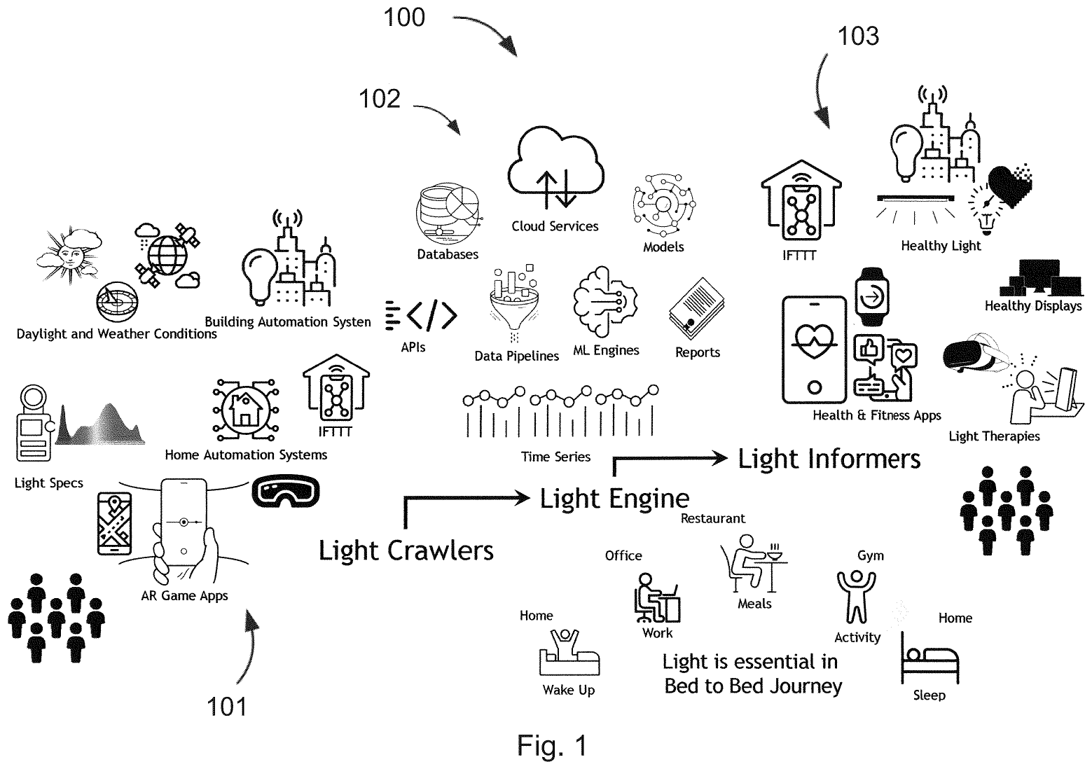

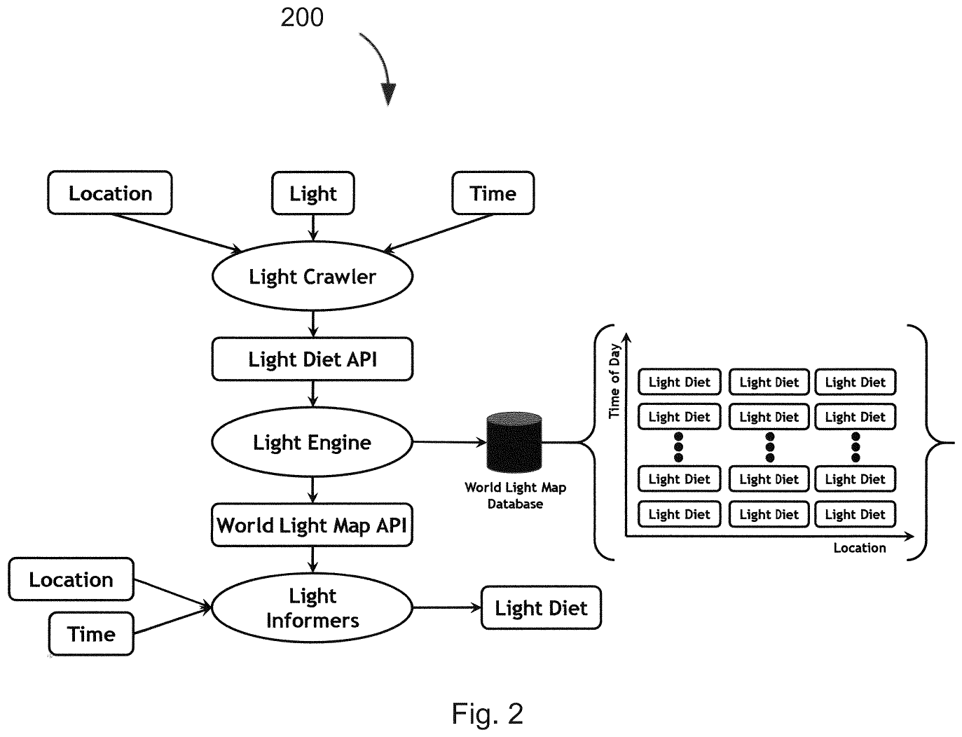

Referring to FIG. 1, a schematic of one embodiment of the WLM platform 100 is shown, and referring to FIG. 2, a flowchart 200 of one embodiment of the method of the WLM platform is shown. This embodiment has three subsystems—Light Crawler 101, Light Engine 102, and Light Informer 103. The Light Crawler 101 is responsible for data collection about the light levels, both quantitative and qualitative measurements for a location. The Light Engine 102 is the central intelligence that combines information from multiple sources, applies the aggregation rules and filtering rules on the light levels, associates with the time of day, and maintains a repository of such records for geo-coordinates. The Light Informers 103 are the APIs that take the inputs from the user on time of day and geo-coordinates to consult the light engine for the corresponding light diet for the specified inputs. Therefore, this system provides the complete workflow of the mapping the light levels for the world.

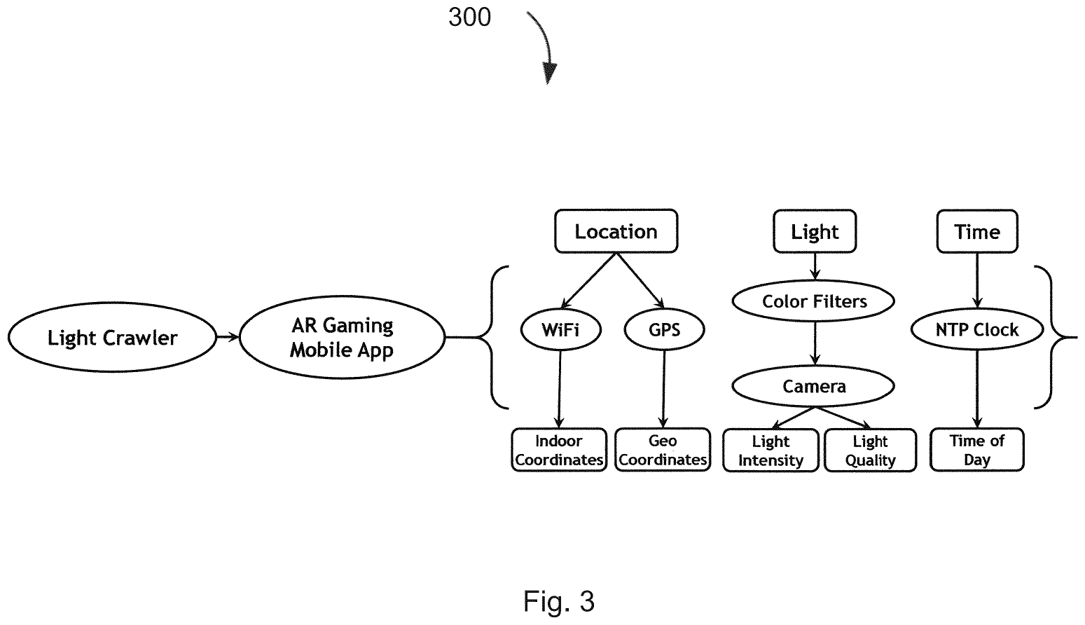

Referring to FIG. 3, a flowchart for one embodiment of the Light Crawlers subsystem is shown. Light Crawlers for artificial light are implemented in the form of, for example, mobile applications, light sensors in a building management system, Light asset management systems, IoT light sensor device in Home Automation, light sensors in Wearables like glasses, watches, rings, buttons or pins and VR Headsets, to name just a few. Light Crawlers, in one embodiment, also measure light based on the sun's position, weather conditions and landscape classification. The sun's position is determined for a geo-location in terms of azimuth and inclination that is specific for a given time of day and day of year. Azimuth and inclination of the sun's position have a strong correlation of the amount of sun light. Weather patterns like an overcast cloudy day, rainy day or clear sunny day have influence on the amount of sun light that gets filtered by the atmosphere. Landscapes such as mountains, thick forests, skyscrapers act as filters or blockers to the sunlight and, therefore, like clouds, are also taken into consideration in one embodiment.

In one embodiment, mobile applications for Light Crawlers can leverage the crowd sourcing approach to gather the light levels where the user walks around indoor space to collect the measurements using the cameras on the phone. For example, in one embodiment, users use game(s) to collect the light levels, translating to a score in the game that can be incentivized for prize, e.g., a healthy light bulb. Such a gam can also be used to generate awareness about the influence of light on one's health in an interactive and innovative gaming approach. Light Crawlers, in one embodiment, can be developed to engage users through gamification of Light Level collection.

Qualitative Measurements, in one embodiment, can be achieved by using low-cost attachments such as, for example, color filters over the cameras on Mobile Phones. These add-ons can also be structured as incentives for the gamers to using it in the Light Crawler games or can be used as trade items between gamers creating a network effect among the social community of gamers.

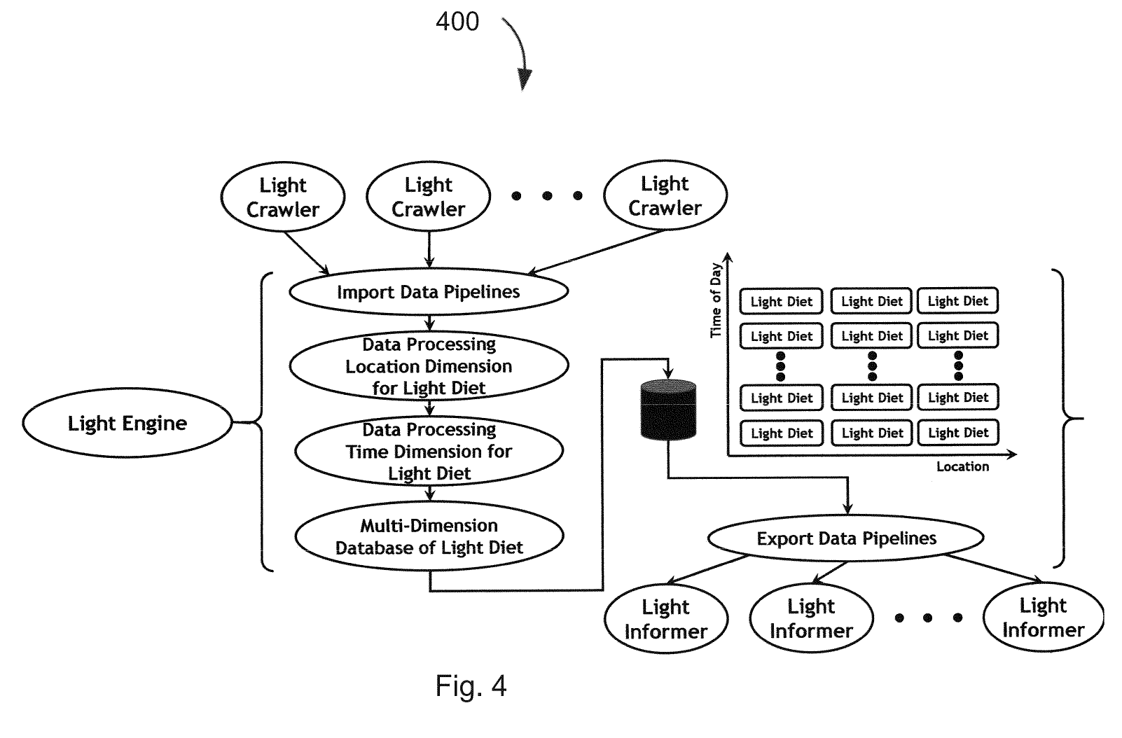

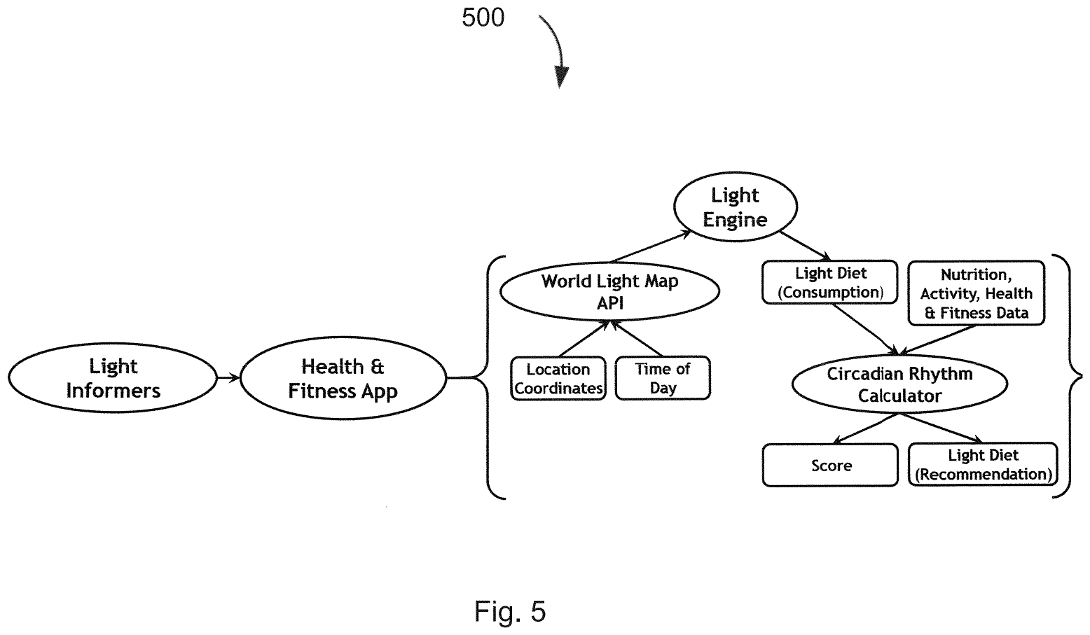

Referring to FIG. 4, a flowchart for one embodiment of the light engine subsystem is shown. Light Engines are the work horse of aggregating the data from the Light Crawlers and indexing the information on a temporal dimension, location dimension among many other dimensions that, in one embodiment, can be extracted through structured learning. In one embodiment, the light engine feeds on a collection of data pipelines, data warehouse and machine learning to derive models for the prediction of light diet for user through Light Informers Referring to FIG. 5, a flowchart for one embodiment of the light informer subsystem is shown. Light Informers are the APIs that offer the ability to integrate into a suite of applications, like Health and Fitness Mobile Applications, Automated Control in Building Management Systems, IFTTT Integrations in Home Automation. Light Informers, in one embodiment, can also be used to calculate a targeted light therapy for the users through a range of deployments like Task Lighting, Light Panels, Picture Mode on Displays, Content for VR Headsets that, in one embodiment, can be used to offset/boost the Light Diet for users.

In one embodiment, the present invention relates to a method of plotting a light map, comprising: (a) gathering interior light data for specific locations inside of buildings in a certain region as a function of time; (b) estimating exterior light data for specific locations outside of building is the certain region as a function of time; (c) wherein the interior and exterior light data includes spectrum and intensity as a function of at least time, and wherein time comprises date and time of day, and wherein accumulated light data comprises the interior and exterior light data; (d) plotting the accumulated light data for the specific locations in the certain region as a function of at least time to create a light map; and (e) wherein the light map provides spectral and intensity data of light as a function of at least time for the specific locations within the certain region.

In one embodiment, the light map changes as a function of time. In one embodiment, the light map changes as a function time and weather. Still other embodiments will be obvious to those of skill the art in light of this disclosure.

In one embodiment, the interior data includes interior space data of a specific location. In one embodiment, the interior space data is a room designation. In one embodiment, the interior space data comprises a user location tag. In one embodiment, the interior light data includes an image of a specific location, and the method further comprises using image recognition to determine the interior space data of the specific location. In one embodiment, the interior light data comprises particular lamp data. In one embodiment, the lamp data is based on a user identifying the lamp being used in a specific location. In one embodiment, the lamp data is based on a lamp in a specific location self-identifying. Still other embodiments will be obvious to those of skill the art in light of this disclosure.

In one embodiment, the exterior light data is calculated based on the Sun's position in terms of azimuth and inclination that is specific for a given time of day and day of year. In one embodiment, the exterior light data is also based on weather at the specific location. In one embodiment, weather is based on a weather pattern such as overcast cloudy day, rainy day or clear sunny day. In one embodiment, the exterior light data is also a function of landscape. In one embodiment, the landscape includes mountains, thick forests, skyscrapers, bodies of water. Still other embodiments will be obvious to those of skill the art in light of this disclosure.

In one embodiment, the spectral data is spectral power density (SPD), and, in another embodiment, the spectral data relates to CCT or a light fidelity index. Light fidelity indices, also known as fidelity indices or color fidelity metrics, are measures used to evaluate how accurately a light source renders colors compared to a reference light source. Here are the most commonly used light fidelity indices: Color Rendering Index (CRI) CRI is a traditional measure of the quality of light in terms of its ability to render colors naturally. It is defined by the International Commission on Illumination (CIE) and uses a scale from 0 to 100, where 100 represents the color rendering of a reference light source. Rf (Fidelity Index): Measures how closely a light source matches the reference light source across 99 color samples. Rg (Gamut Index): Indicates the average increase or decrease in chroma (color saturation). Still other embodiments will be obvious to those of skill the art in this exposure.

In one embodiment, the light data is gathered using light crawlers. In one embodiment, the light crawlers include at least one of Light Crawler for artificial light implemented in the form of at least one of mobile applications, light sensors in a building management system, light asset management systems, IoT light sensor device in Home Automation, light sensors in wearables like glasses, watches, rings, buttons or pins and VR Headsets. In one embodiment, the light crawlers include at least one of Light Crawler for natural light track sun's position, weather conditions and landscape classification.

In one embodiment, light crawlers comprise smart phone with cameras and mobile applications that leverage crowd sourcing to gather the light levels where the user walks around indoor space to collect the measurements using the cameras on the phone. In one embodiment, the mobile application is a game for users to collect the light levels translating to a score in the game.

In one embodiment, the light data is plotted using a Light Engine, the light engine aggregates the light data from the Light Crawlers and indexes the information on at least a temporal and location dimension.

In one embodiment, the present invention relates to a method of prompting a user to obtain a light requirement, comprising: (a) based on a user's presence in one or more specific locations within a certain region, determining a user's light exposure to light based a light map which provides spectral and intensity data of light as a function of at least time for the specific locations within the certain region; (b) determining a light prescription for the user based at least in part on the user's circadian rhythm; (c) determining the user's light requirement based on the light prescription and the light exposure; and (d) prompting the user to be exposed to the light requirement to satisfy the light prescription.

In one embodiment, the light requirement is the light prescription minus the light exposure.

In one embodiment, a light informer comprises an API to integrate a system to provide the light prescription.

In one embodiment, the system is at least one of a health and fitness mobile application, an automated control in building management systems, a healthy display, a healthy light, a light therapy system, or an IFTTT integration in home automation.

In one embodiment, the method of prompting a user to obtain a light requirement, further comprising: providing light to the user to deliver the light prescription, and/or directing the user to a specific location for a certain duration to be exposed to the light prescription.

In one embodiment, the exposure is measured in day light hours. In one embodiment, the exposure is calculated using a light engine.

In one embodiment, the present invention relates to a method of enhancing light in a specific location, comprising: (a) gathering light data for specific locations in a certain region as a function of time; (b) wherein the light data includes spectrum and intensity as a function of at least time, and wherein time comprises date and time of day; (c) plotting the light data for the specific locations in the certain region as a function of at least time to create a light map; (d) wherein the light map provides spectral and intensity data of light as a function of at least time for the specific locations within the certain region; (e) determining a lighting requirement of at least one specific location based on at least a function of the at least one specific location; and (f) recommending a modification in light at the at least one specific location based on the light map and the lighting requirement. In one embodiment, the recommendation is made only to an owner of the at least one specific location.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein and are intended to be within the spirit and scope of the invention. Accordingly, the foregoing description is by way of example only, and not limiting. The invention is limited only as defined in the following claims and equivalents thereto.

What is claimed is:

1. A method of plotting a light map comprising:
gathering interior light data for specific locations inside of buildings in a certain region as a function of time;
estimating exterior light data for specific locations outside of building is said certain region as a function of time;
wherein said interior and exterior light data includes spectrum and intensity as a function of at least time, and wherein time comprises date and time of day, and wherein accumulated light data comprises said interior and exterior light data;
plotting said accumulated light data for said specific locations in said certain region as a function of at least time to create a light map; and
wherein said light map provides spectral and intensity data of light as a function of at least time for said specific locations within said certain region.

2. The method of claim 1, said light map changes as a function of time or as a function of time and weather.

3. The method of claim 1, said interior data includes interior space data of a specific location.

4. The method of claim 3, said interior space data comprises at least one of a room designation, a user location tag, lamp data, or an image of a specific location in which case said method further comprises using image recognition to determine said interior space data of said specific location.

5. The method of claim 4, said lamp data is based on a user identifying the lamp being used in a specific location.

6. The method of claim 1, wherein said exterior light data is calculated based on the Sun's position in terms of azimuth and inclination that is specific for a given time of day and day of year.

7. The method of claim 6, wherein said exterior light data is also based on weather at said specific location.

8. The method of claim 1, wherein said exterior light data is also a function of landscape.

9. The method of claim 1, wherein said spectral data is spectral power density (SPD).

10. The method of claim 1, wherein said light data is gathered using light crawlers.

11. The method of claim 10, wherein said light crawlers include at least one of Light Crawler for artificial light implemented in the form of at least one of mobile applications, light sensors in a building management system, light asset management systems, IoT light sensor device in Home Automation, light sensors in wearables like glasses, watches, rings, buttons or pins and VR Headsets.

12. The method of claim 11, wherein said light crawlers include at least one of Light Crawler for natural light track sun's position, weather conditions and landscape classification.

13. The method of claim 11, wherein light crawlers comprise smart phone with cameras and mobile applications that leverage crowd sourcing to gather the light levels where the user walks around indoor space to collect the measurements using the cameras on the phone.

14. The method of claim 13, wherein said mobile application is a game for users to collect the light levels translating to a score in the game.

15. The method of claim 10, wherein the light data is plotted using a Light Engine, said light engine aggregates the light data from the Light Crawlers and indexes the information on at least a temporal and location dimension.

16. A method of prompting a user to obtain a light requirement, comprising:
based on a user's presence in one or more specific locations within a certain region, determining a user's light exposure to light based a light map which provides spectral and intensity data of light as a function of at least time for said specific locations within said certain region;
determining a light prescription for said user based at least in part on said user's circadian rhythm;
determining said user's light requirement based on said light prescription and said light exposure; and
prompting said user to be exposed to said light requirement to satisfy said light prescription.

17. The method of claim 16, wherein said light requirement is said light prescription minus said light exposure.

18. The method of claim 16, wherein a light informer comprises an API to integrate a system to provide said light prescription.

19. The method of claim 16, further comprising: providing light to said user to deliver said light prescription.

20. A method of enhancing light in a specific location, comprising:
gathering light data for specific locations in a certain region as a function of time;
wherein said light data includes spectrum and intensity as a function of at least time, and wherein time comprises date and time of day;
plotting said light data for said specific locations in said certain region as a function of at least time to create a light map;
wherein said light map provides spectral and intensity data of light as a function of at least time for said specific locations within said certain region;
determining a lighting requirement of at least one specific location based on at least a function of said at least one specific location; and
recommending a modification in light at said at least one specific location based on said light map and said lighting requirement.
